(19) United States
(12) Patent Application Publication
(10) Pub. No.: US 2003/0186935 A1
(43) Pub. Date: Oct. 2, 2003

(54) Myeloglycan

(76) Inventors: Kazuko Handa, Seattle, WA (US); Mark R. Stroud, Seattle, WA (US); Steven B. Levery, Seattle, WA (US); Tatsushi Toyokuni, Seattle, WA (US); Sen-Itiroh Hakomori, Mercer Island, WA (US)

Correspondence Address: GRAY CARY WARE FREDENRICH, 1625 MASSACHUSETTS AVENUE, NW, SUITE 300, WASHINGTON, DC, 20036-2247, US

(21) Appl. No.: 10/326,821

(22) Filed: Apr. 25, 2003

Related U.S. Application Data
(63) Continuation of application No. 09/516,497, filed on Mar. 1, 2000, now abandoned.
Application No. 09/516,497 is a continuation of application No. 08/435,664, filed on May 5, 1995, now abandoned.
Application No. 08/435,664 is a continuation-in-part of application No. 08/353,328, filed on Dec. 5, 1994, now abandoned.

(51) Int. Cl. A61K 31/739; C08B 37/00

(52) U.S. Cl. 514/54; 536/53

(57) ABSTRACT

Systematic chemical analysis of glycosphingolipid (GSL) fractions from large quantities of normal human neutrophils and HL60 cells failed to detect GSL's containing an SLe^{x} structure. Instead, the binding target of E-selectin was revealed to be a series of long-chain, unbranched polylactosamine GSL's with a terminally sialylated, internally polyfucosylated structure, called, “myeloglycan”.

This is a continuation of U.S. patent application Ser. No. 08/435,664, which is a continuation-in-part patent application of U.S. patent application Ser. No. 08/353,328, now abandoned, the entire contents of each are incorporated herein by reference.

Portions of the research described herein were supported in part by a grant from the National Institutes of Health.

### BACKGROUND OF INVENTION

E-selectin and P-selectin are expressed on activated endothelial cells (EC's). P-selectin also is expressed on activated platelets. Both selectins play roles in various phases of cell interactions, such as, the inflammatory response.

P-selectin is localized at (i) Weibel-Pallade bodies present in the cytoplasm of resting EC's and (ii) α-granules of resting platelets. When EC's or platelets are activated by various factors (e.g. thrombin, ADP, phorbol esters, histamine and free radical oxygen [O₂—]), Weibel-Pallade bodies or α-granules are translocated rapidly to the EC or platelet surface, leading to P-selectin expression. The exact mechanism of such translocation is not well understood, but likely involves a number of transmembrane signaling mechanisms, e.g. those mediated by protein kinase C, thromboxane and eicosenoids. The translocation/expression process is rapid (takes only 1-3 minutes).

In contrast, expression of E-selectin at the EC surface, which results, for example, from stimulation by TNFα and IL-1β, requires de novo synthesis of E-selectin, i.e. a 4-5 hour “lag time” between stimulation and expression.

P-selectin is believed to be involved in the initial rapid adhesion of neutrophils to EC's, while E-selectin is believed to be involved in subsequent reinforcement of that adhesion. Both processes are important in mediation of the inflammatory response.

E-selectin and P-selectin-mediated adhesion of neutrophils to EC's is considered to be an important step in the process of neutrophil recruitment and accumulation at inflammatory sites resulting from wounding, infection, or blocking of blood circulation (thrombosis); The major damage from the inflammatory response results from accumulation of neutrophils which produce O₂— and H₂O₂, which in turn cause serious tissue damage. For example, the major tissue damage following heart attack or brain hemorrhage (stroke) results from neutrophil migration and accumulation in tissues, rather than from ischemia (blocking of blood supply). An example is the “reperfusion injury” which occurs when a thrombosis is eliminated by specific treatment and blood circulation is restored. As a consequence of reperfusion, many neutrophils migrate out of the capillaries into surrounding tissues, damaging tissue structure and function.

Immediately after the overall sequence of selectins was clarified through cDNA cloning, and the presence of a C-type lectin domain at the N-terminal domain of both P-selectin and E-selectin was demonstrated (for example, 1 and 2), many workers undertook an intensive search for the carbohydrate epitopes recognized by those selecting.

SLe^{x} has been considered to be a plausible ligand of P-selectin and E-selectin based on the following observations: (i) transfection of Lewis fucosyltransferase cDNA to Chinese hamster ovary (CHO) cells expressing sialosyl type 2 chain resulted in acquisition of the ability to adhere to TNFα-activated endothelial cells (3). (ii) HL60 cells, previously shown to react with mAb FH6, are capable of binding to TNFα-activated or IL-1-activated EC's, and the binding can be inhibited by liposomes containing SLe^{x}-bearing GSL's but not by liposomes containing sialosylparagloboside, sialosylnorhexaosylceramide or Le^{x}-glycosylceramides. (iii) mAb's SNH3 and SNH4 inhibited E-selectin-dependent HL60 cell adhesion (4). (iv) Subsequent confirming studies utilized other anti-SLe^{x} mAb's, oligosaccharides or GSL's containing the SLe^{x} structure.

Some studies indicated that selectin-dependent binding, particularly in tumor cells, also is mediated by SLea (a positional isomer of SLe^{x}) (5-7). However, SLe^{a}, which has a lacto-series type 1 chain structure, is completely absent from human neutrophils and HL60 cells.

Based on antibody reactivity, SLe^{x} is thought to be expressed in the form of O-linked, N-linked or lipid-linked carbohydrate chains.

Although many selectin-related studies since have been published, those studies all were based on inhibition by or adherence to only a suspected structure. There has been almost no effort directed to elucidating the chemical isolation and characterization of the real carbohydrate target structure of selectins present in normal human neutrophils or HL60 cells, because of the extreme difficulty of isolating and characterizing the essential epitope expressed in those cells.

Tiemeyer et al. (8) isolated the VIM-2 antigen structure from a relatively large quantity of HL60 cells. VIM-2 has the structure,

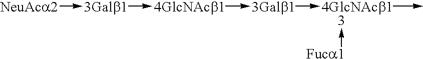

and was believed to be the E-selectin binding site. However, Lowe et al. (9) failed to observe E-selectin-dependent adhesion of VIM-2-positive, SLe^{x}-negative CHO cells and therefore were unable to confirm the role of VIM-2 role in E-selectin-dependent cell adhesion.

Using large quantities of HL60 cells (≧1000 mL packed) and human neutrophils (≈100 mL packed), the instant inventors investigated structures binding to E-selectin expressed on CHO cells. Contrary to previous suggestions, the binding site was identified as a series of novel unbranched long-chain sialylated polylactosamine (PLA) internally polyfucosylated structures, hereby collectively termed “myeloglycan”. VIM-2 antigen did not bind to E-selectin. Neither SLe^{x}, bivalent SLe^{x}, sialosyl dimeric Le^{x} nor sialosyl trimeric Le^{x} were present in neutrophils or HL60 cells. Therefore, none of those structures are physiological ligands of E-selectin in lymphocytes.

### SUMMARY OF THE INVENTION

The instant invention relates to a class of isolated novel unbranched, long chain, 2→3 sialylated, internally α1→3 fucosylated, except at the penultimate N-acetyl glucosamine, polylactosamines.

The instant invention also contemplates a class of isolated novel unbranched, long chain, 2→3 sialylated, internally α1→3 fucosylated polylactosamines, wherein the penultimate N-acetyl glucosamine is fucosylated.

The instant invention also relates to use of such isolated unbranched, long chain, sialylated, internally fucosylated polylactosamines, or derivatives thereof, to intervene in selectin-mediated phenomena.

Moreover, the instant invention relates to methods for making myeloglycans and derivatives thereof.

### BRIEF DESCRIPTION OF THE DRAWINGS

FIGS. 1A and 1B present HPTLC profiles of the HL60 cell monosialoganglioside fraction separated by HPLC on an Iatrobead™ column.

FIG. 1A: The monosialoganglioside fraction was prepared from 300 mL of packed HL60 cells as described herein. The fraction was mixed with 500 μL of isopropanol: hexane: water (IHW), 55:40:5, v/v/v, sonicated and injected onto an Iatrobead™ column (6RS-8010, 0.4×30 cm) pre-equilibrated with IHW, 55:40:5. Gradient elution from that solvent to IHW, 55:25:20, was performed over 400 min at a flow rate of 0.5 mL/min. Two mL fractions were collected and a 5 μL sample from each fraction was spotted on high performance thin layer chromatography (HPTLC) silica gel plates (EM Science, Gibbstown, N.J.). HPTLC was developed with chloroform/methanol/0.5% CaCl₂ (50:55:19), and bands were revealed by reaction with an orcinol-sulfuric acid reagent. A, B, and C denote TLC migration positions of (respectively) three types of SLe^{x} GSL:

NeuAcα2→3Galβ1→4[Fucα1→3]GlcNAcβ1→3Galβ→4Glcβ→1Cer,

NeuAcα2→3Galβ1→4[Fucα1→3]GlcNAcβ1→3Galβ1→4GlcNAcβ1→3 Galβ1→4Glcβ1→1Cer and

NeuAcα2→3Galβ1→4[Fucα1→3]GlcNAcβ1→3Galβ1→4[Fucα1→3]GlcNAcβ1→3Galβ1→4Glcβ1→1Cer.

FIG. 1B: The polar monosialoganglioside fraction of HL60 cells was separated on HPLC in the IHW solvent system as described herein. Bands were revealed by TLC blotting with E-selectin-expressing CHO cells metabolically labeled with ³²p (14). Lanes 1-16 correspond respectively to fractions 9, 19, 21, 27, 31, 33, 37, 39, 41, 43, 44, 45, 46, 47, 48 and 49 of FIG. 1A. The right-hand lane is SLe^{x} ceramide hexasaccharide. All E-selectin binding fractions were slow-migrating GSL's containing long-chain PLA. No band was eluted corresponding to an SLe^{x}containing GSL (see FIG. 1A), although those species are found abundantly in eluates from human carcinoma tissues (15). Major reactivity with E-selectin was observed in very polar fractions, beginning with fraction 43 (lane 10).

FIG. 2 presents a comparison of E-selectin-binding monosialoganglioside fractions extracted from human neutrophils and HL60 cells.

About 100 mL of human neutrophils were extracted and the monosialoganglioside fraction thereof was prepared as described herein. The fraction was compared with a corresponding fraction prepared from HL60 cells by HPTLC followed by blotting with ³²P-labeled E-selectin-expressing CHO cells (14). Lane 1, SLe^{x} ceramide hexasaccharide. Lane 2, total Folch's upper-layer GSL's from HL60 cells. Lane 3, purified-monosialoganglioside fraction from lane 2. Lane 4, purified monosialoganglioside fraction from Folch's upper-layer GSL's from human neutrophils. The quantity of ganglioside mixture used for lanes 3 and 4 was based on approximately equal numbers of HL60 cells and neutrophils. Lanes 5 and 6, same as lanes 3 and 4 but diluted 2×.

FIGS. 3A-3F depict reactivity of myeloglycans, before and after sialidase treatment, with various mAb's.

For each figure, lane 1, fraction 12.2; lane 2, fraction 12.2 after sialidase treatment; lane 3, fraction 13.1; lane 4, fraction 13.1 after sialidase treatment; lane 5, dimeric Le^{x} (III³FucV³FucnLc₆); and lane 6, nLc₆. FIG. 3A: immunoblotting with anti-Galβ1→4GlcNAcβ1→3Gal mAb 1B2. FIGS. 3B-3D: immunoblotting with anti-Le^{x} mAbs, SH1, FH2 and anti-SSEA-1, respectively. FIG. 3E: immunoblotting with mAb PL82G2. FIG. 3F: glycolipid bands revealed by reaction with an orcinol-sulfuric acid reagent.

Sialidase treatment of fractions 12.2 and 13.1 was performed by incubation of 1 μg of glycolipid dissolved in 20 μL of 0.1 M sodium acetate (pH 4.5) containing 0.02 units Clostridium perfringens sialidase at 37° C. for 2 hr. Five μL of the reaction mixture was spotted onto a TLC plate and washed with water. The plate was dried and developed for 20 min in C:M:CaCl₂ (50:55:19).

FIGS. 4A and 4B depict ¹H-NMR spectra of myeloglycans that bind (fraction 14; FIG. 4B) or do not bind (fraction 13-0; FIG. 4A) to E-selectin.

The two spectra are characterized by several common features: (i) α-anomeric signal at 4.875 ppm, diagnostic for Fucα1→3 linked to type 2 chain GlcNAcβ1→3 residue. (ii) A broadened and distorted quartet assignable to H-5 of the same Fucα1→3 substitution. (iii) A duplet at 1.015 ppm assignable to the Fucα1→3 methyl group (H-6). (iv) Duplets at 2.576 ppm for H-3_{eq} of terminal NeuAcα2→3 (32). (v) A singlet at 1.889 ppm for the N-acetyl methyl group of NeuAcα2→3. (vi) A β-anomeric signal at 4.174 ppm assignable to Glcβ1→1Cer.

In contrast, there are clear differences between spectra of E-selectin non-binding fraction 13-0 and binding fraction 14: (i) Compared to fraction 13-0, fraction 14 has a much more intense (2-3 times higher) signal at 4.875 ppm. (ii) The GlcNAc-1 signals at 4.736 ppm (assigned as VII-1) and 4.748 ppm (assigned as IX-1) were prominent for fraction 14 but absent or unclear for fraction 13-0. (iii) Fraction 14 showed greater upfield shifting of the GlcNAc-1 resonance and gave a more complex pattern, that is, the presence of resonances at 4.748, 4.736, and 4.700 ppm, which may be assignable to 3-substituted GlcNAc-1. The presence in fraction 13-0 of a duplet at 4.741 ppm likely is due to 3-substituted GlcNAc-1. (iv) The quartet assignable to H-5 of the Fucα1→3 substituent shows a more complex pattern in the spectrum of fraction 14 than of 13-0. The spectrum assignable to Gal-1 was more complex and broadened in fraction 14 than in 13-0, suggesting complex interaction with the internal substituent.

FIG. 5 depicts part of a myeloglycan including the repeating GlcNAc-Gal subunit. Below the backbone are various groups which can substitute for the sialyl residue at R¹ and various groups which can substitute for a fucosyl residue at R².

FIG. 6 depicts a synthetic scheme for obtaining a starting material (4) in the chemical synthesis of myeloglycan. EtSH is mercaptoethanol. Ac is the acetyl group. MeOH is methanol. NaOMe is sodium methoxide. Bu₂SnO is dibutyltin oxide.

FIG. 7 depicts a scheme for the chemical synthesis of Le^{x} derivatives containing CF₃-Fuc or 5-S-Fuc. BF₃-Et₂O is boron trifluoride diethyl etherate.

FIG. 8 depicts a scheme for the chemical synthesis of Le^{x} derivatives containing 1-S-Fuc or C-Fuc. DMSO is dimethylsulfoxide. NaBH₄ is sodium borohydride. pyr is pyridine.

FIG. 9 depicts a continuation of the scheme depicted in FIG. 8 wherein the triflate is treated to yield the desired products.

FIG. 10 depicts a scheme for attaching the various derivatives to a linear linking molecule or tether.

FIG. 11 depicts a scheme for synthesizing dimeric and trimeric Le^{x} derivatives. (Ph₃P)₃RhCl is tris(triphenylphosphine)rhodium(I) chloride. DBU is 1,8-diazabicyclo[5.4.0]undec-7-ene. MeOTf is methyl trifluromethanesulfonate.

FIG. 12 depicts a scheme for the synthesis of the core of myeloglycan. Ac₂O is acetic anhydride.

FIG. 13 depicts a scheme for the synthesis of myeloglycan. CrO₃ is chromium(VI) oxide. HBBr₂.SMe₂ is dibromoborane-methyl sulfide complex. Pd/C is palladium on carbon. SO₃.NMe₃ is a complex of sulfur trioxide and trimethylamine.

FIG. 14 depicts schemes for synthesizing a multivalent myeloglycan structure. Boc₂O is di-tert-butyl dicarbonate. DCC is 1,3-dicyclohexylcarbodiimide. TFA is trifluoroacetic acid.

FIG. 15 depicts alternative methods for obtaining polyvalent myeloglycan structures by incorporation into a liposome (top) or by polymerization (bottom). The symbols are as provided in earlier legends.

### DETAILED DESCRIPTION OF THE INVENTION

As used herein, “isolated” indicates some level of intervention wherein biologically active molecules in situ are removed from the naturally occurring situs. Generally, isolation involves a level of purification.

Also, “cell” in meant to indicate a biologic entity that carries myeloglycan or selectin at the surface thereof. The cell may or may not contain a nucleus.

The myeloglycans of the instant invention comprise a discrete class of carbohydrate found in, for example, cells of the immune system.

Neither HL60 cells nor human neutrophils expressed GSL's containing an SLe^{x} terminal epitope as isolated previously and characterized from human colonic and other carcinoma tissues (15,16). Many E-selectin-binding components eluted on HPLC were slow-migrating, extremely polar GSL's, some of which were characterized as having unbranched long-chain PLA backbone structures with a minimum of 4 N-acetyllactosamine subunits. The existence of pairs of structures, one binding to E-selectin, the other not (e.g. fractions 12 vs. 13-1 and 13-0 vs. 14) indicates that E-selectin binding is based on terminally α2→3 sialylated, internally multiply fucosylated structures. A sulfate group is not involved in the physiological process of neutrophil binding to E-selectin. Analysis of GSL fractions of HL60 cells and neutrophils indicates that myeloglycan (but not SLe^{x}) is the physiological E-selectin binding epitope.

Suitable cells for obtaining myeloglycans are those known to express ligands which bind selectin expressed on, for example, endothelial cells and platelets. Thus, cells of the immune system, which are known to bind to activated endothelium, for example, and specifically, which bind by virtue of reacting with selectin, are likely to contain myeloglycans and are suitable starting materials. Accordingly, lymphocytes, such as neutrophils, and various publicly available cell lines of immune cell origin can be used to isolate myeloglycan.

The cells are isolated using known techniques, such as centrifugation of whole blood, passing blood through an affinity matrix containing a reagent which can capture the cells of interest, for example, an antibody specific to a cell surface molecule on the target cell, and the like.

Alternatively, cell lines are cultured using known methods and reagents. The cells are passed at appropriate intervals and collected by centrifugation.

The highly polar glycosphingolipids (GSL's) of the cells are extracted by exposing lysed cells, for example, following exposure to freezing temperatures, in a solvent, such as a mixture of an alcohol, an organic liquid and an aqueous liquid. A suitable solvent is one which can be used in a gradient elution chromatographic procedure. A suitable alcohol is isopropanol (I), a suitable organic liquid is hexane (H) and a suitable aqueous liquid is water (W). A suitable solvent is IHW in a ratio of 55:50:25, v/v/v.

The cells are extracted repeatedly with a suitable volume of solvent. The extraction can be assisted using a mortar and pestle or an electric blender. The fluid phase is passed through a filter to remove the particulate matter, such as by filtering through diatomaceous earth.

The extracts are combined and evaporated to dryness.

The residue is dissolved in a volume of an aqueous solvent, such as water. The resulting solution was Folch partitioned with six volumes of chloroform (C): methanol (M), 2:1, v/v. The lower phase is repartitioned repeatedly with theoretical upper phase.

The upper phases are combined, the volume is reduced to a small volume, such as, about 10 ml, for example, by evaporation, and the sample is dialyzed against an aqueous buffer, such as distilled water, using dialysis tubing with a molecular weight cut-off of about 5000.

The dialysate is lyophilized and dissolved in a suitable liquid solvent in preparation for chromatographic separation, such as, chloroform (C):methanol (M):water (W), as described in (11). Thus, a suitable buffer is CMW at a ratio of 1:10:10, v/v/v. The solution is passed over a DEAE column, for example, having dextran as the inert carrier.

The monosialoganglioside fraction is eluted using the same solvent, for example, the 1:10:10 CMW solvent, but containing 0.03 M ammonium acetate.

The various monosialogangliosides can be separated on adsorption to, for example, a silica gel matrix. A suitable matrix is IATROBEADS™, and a suitable solvent is IHW, as taught in (12 and 13). Hence the starting solvent can have a component ratio of 55:40:5, v/v/v of IHW, and elution occurs over a period of about seven hours at a flow rate of about 0.5 ml per minute wherein the solvent gradient varies to a final composition of, for example, 55:25:20 of IHW, to obtain separation, as known in the art. As noted in the drawings herein, essentially pure species of monosialogangliosides can be obtained.

The various species can be separated further by acetylation and preparative high performance thin layer chromatography as described in (12) and (13).

Determination of whether a monosialoganglioside binds selectin can be accomplished in any of a variety of art-recognized means. For example, (14) teaches a blotting-type method wherein the separated species are exposed to labelled cells known to express selectin, such as activated endothelial cells.

It was determined that the sialyl Le^{x} (SLe^{x}) structure does not have a role as a selectin ligand in immune cells and HL60 cells. That conclusion was obtained on analysis of the various species of sugars isolated, as described herein, from HL60 cells (obtained from the ATCC) which are known to bind to activated endothelium via selectin.

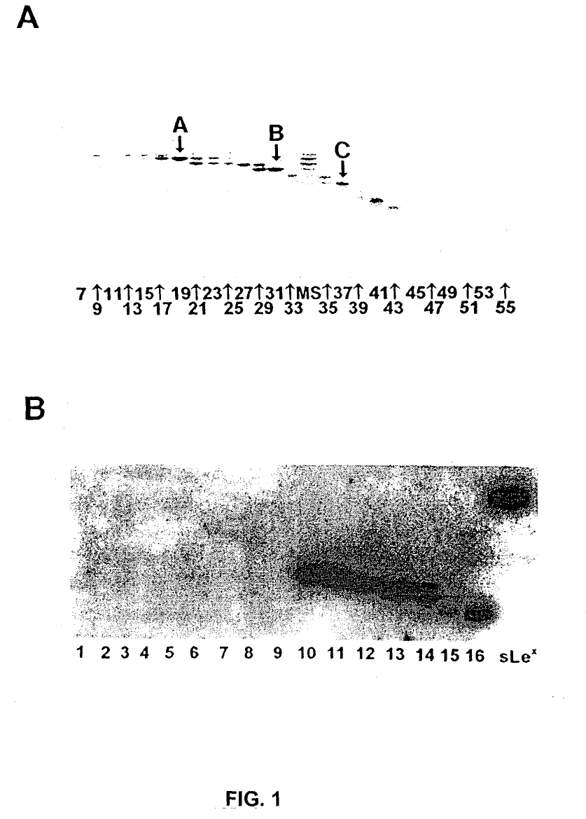

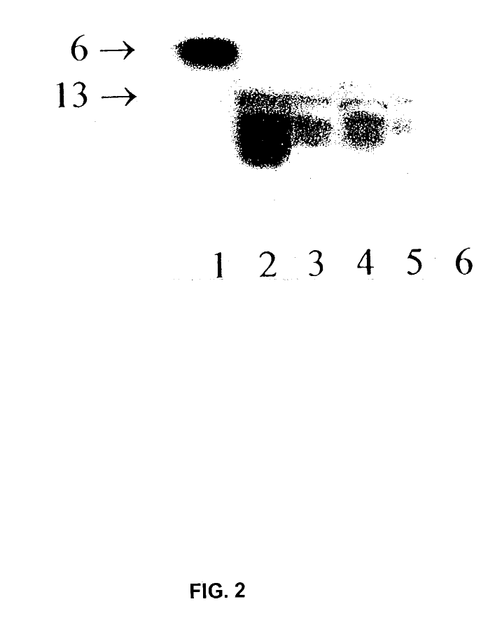

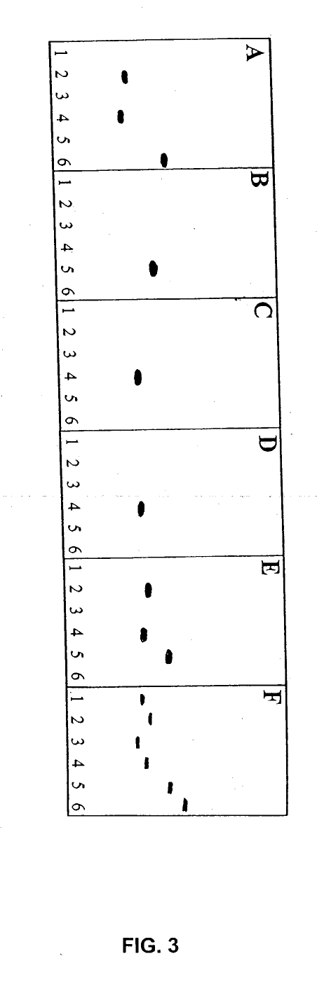

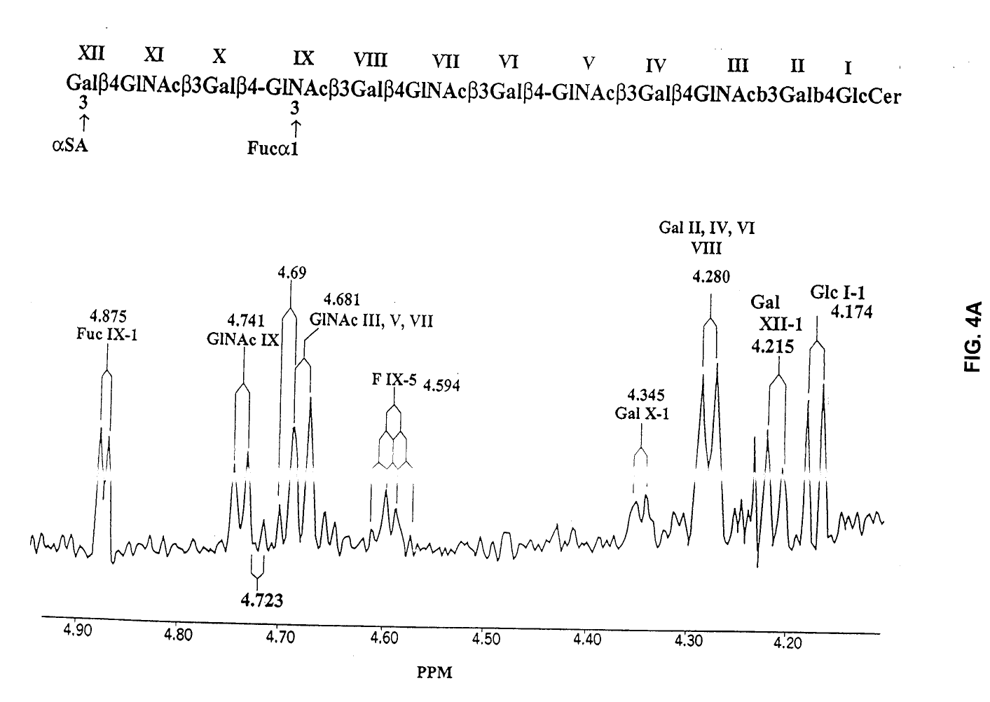

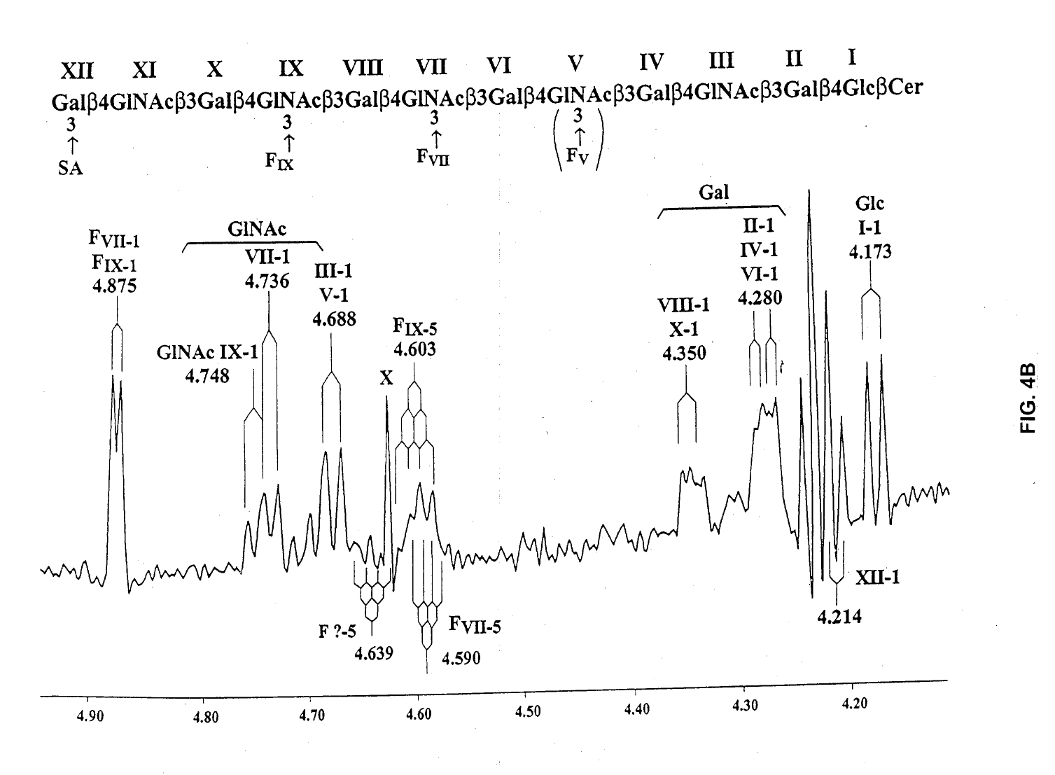

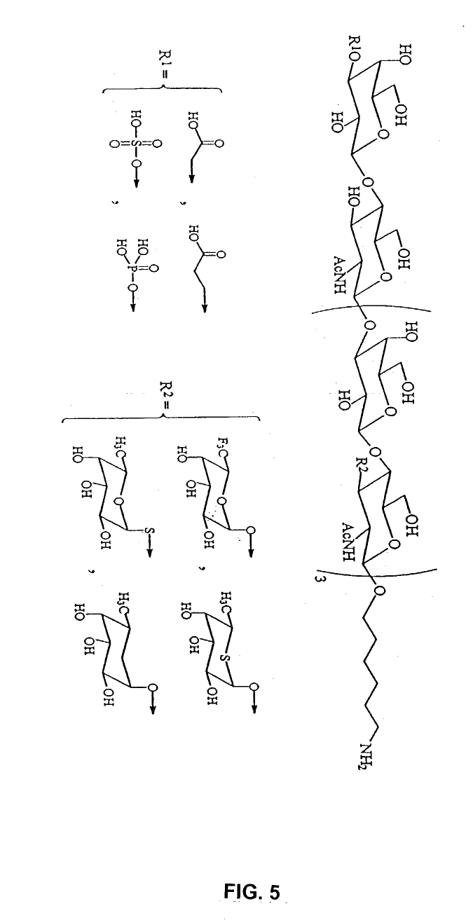

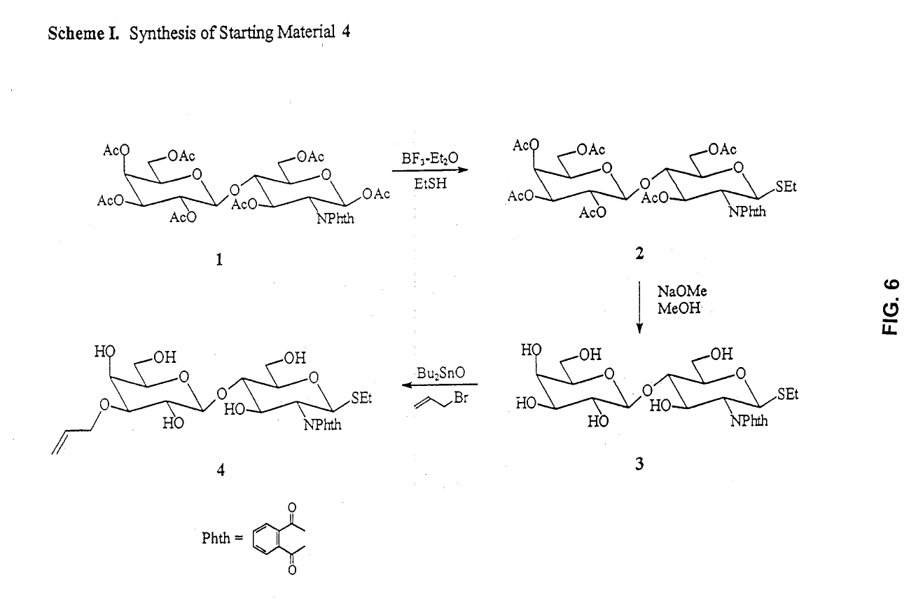

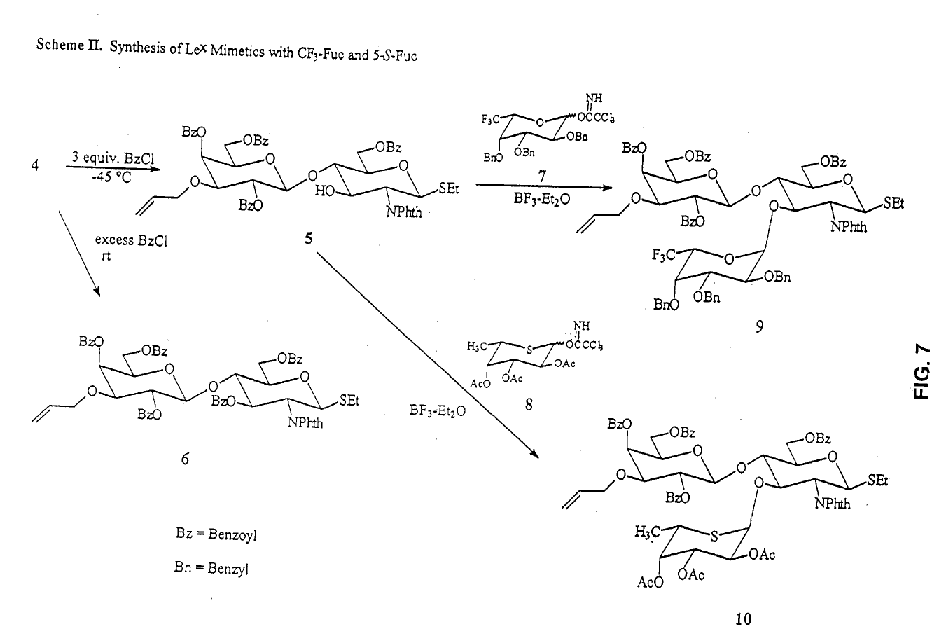

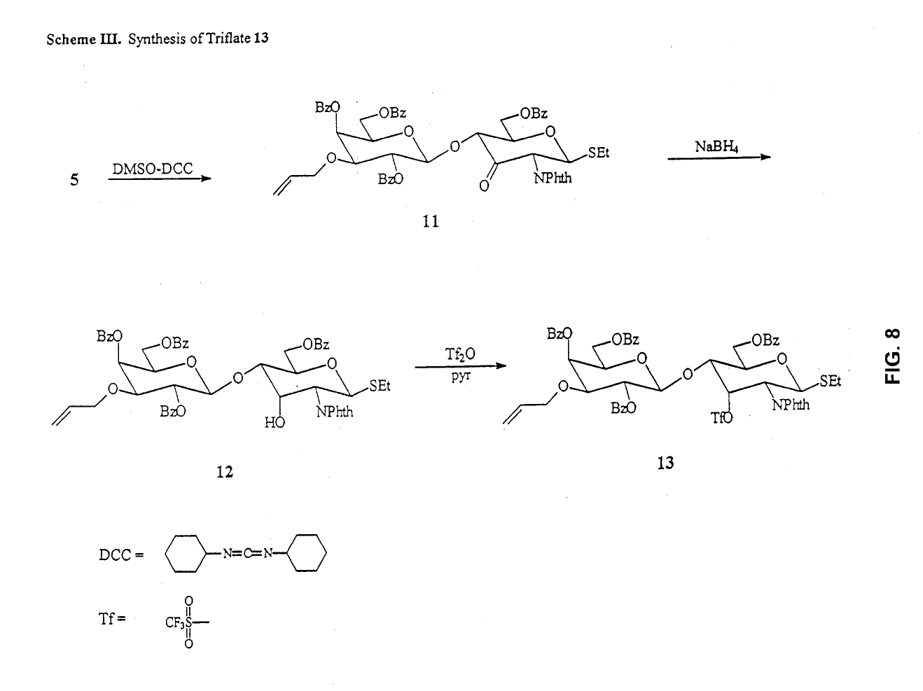

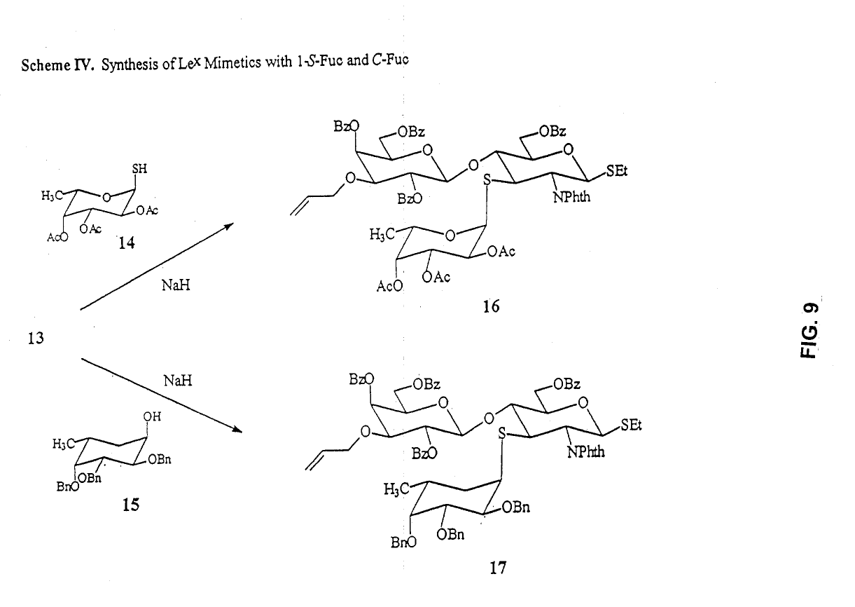

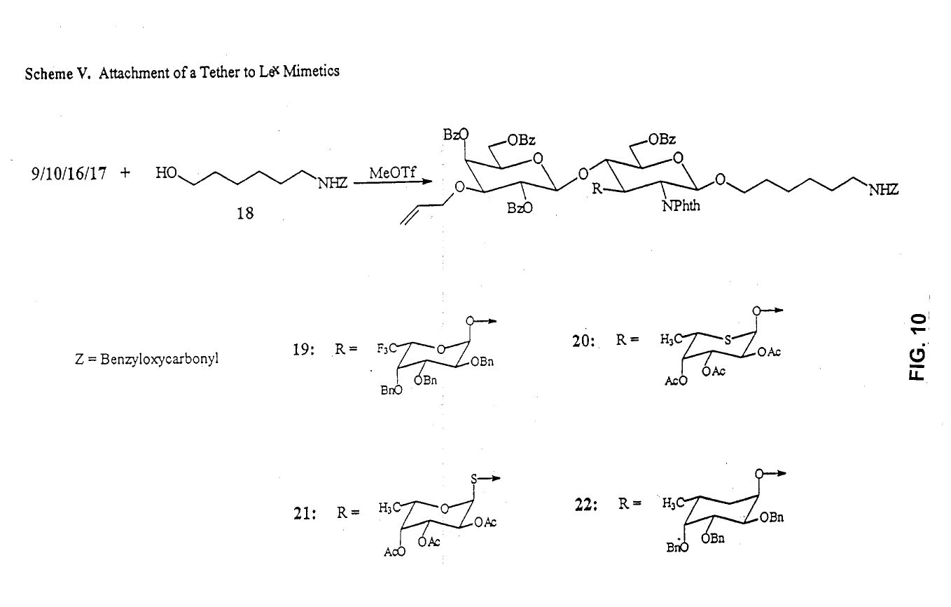

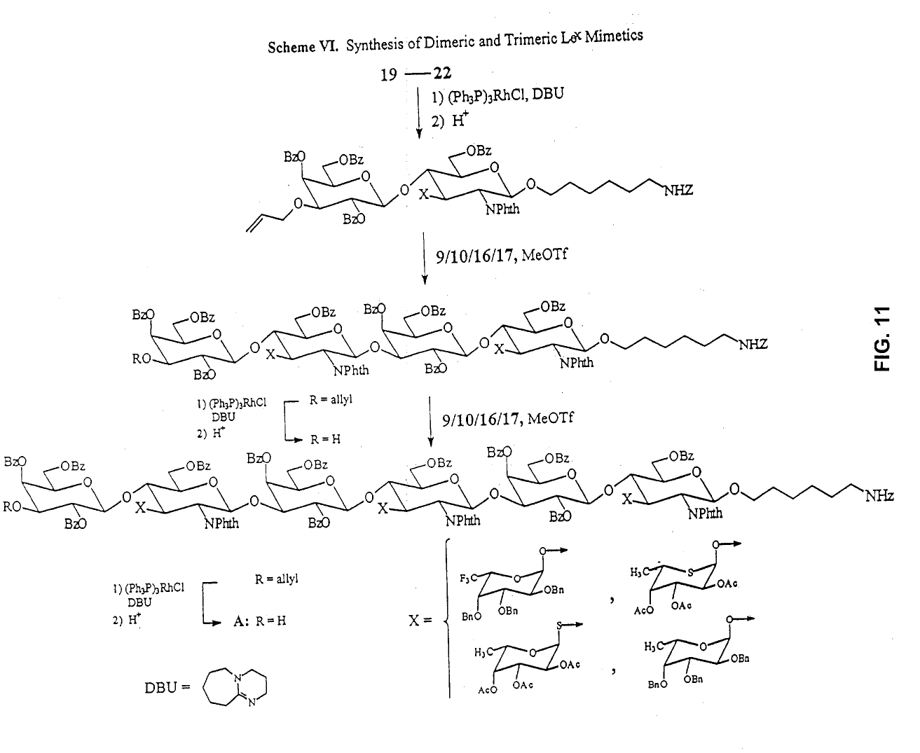

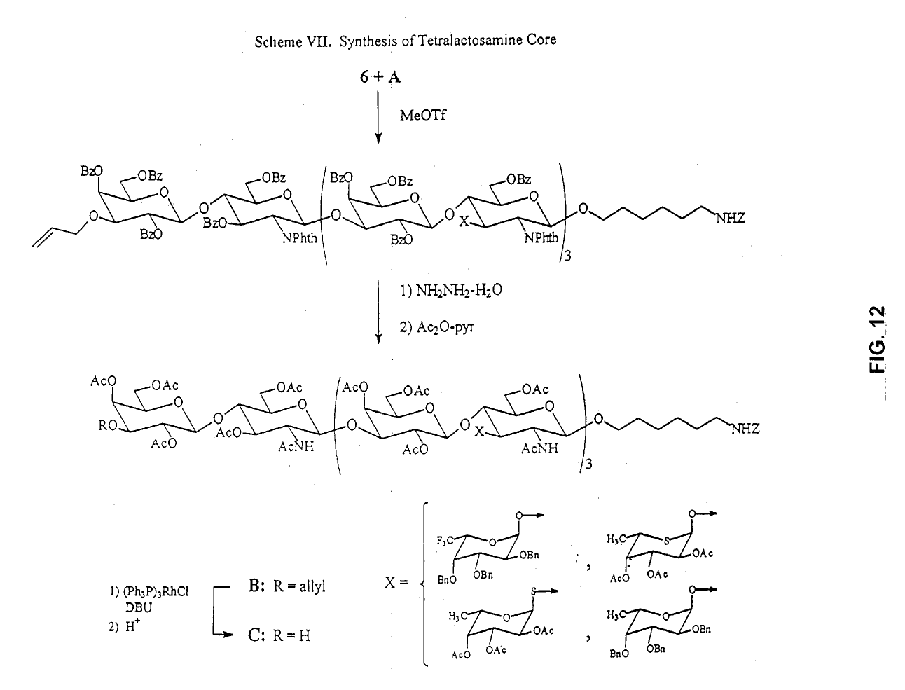

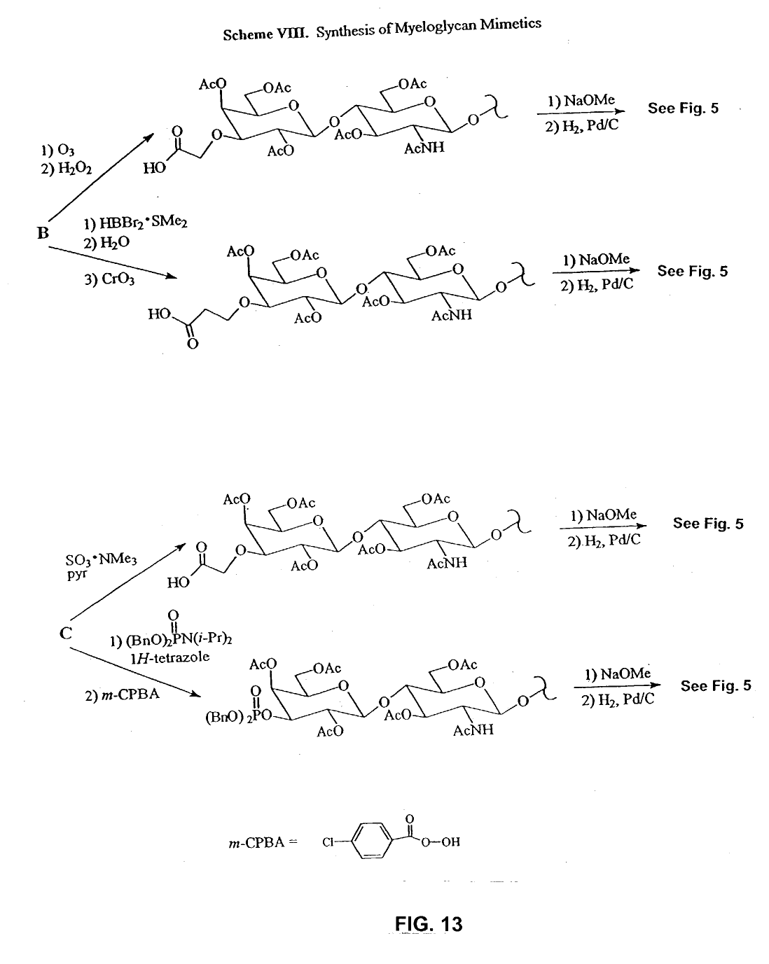

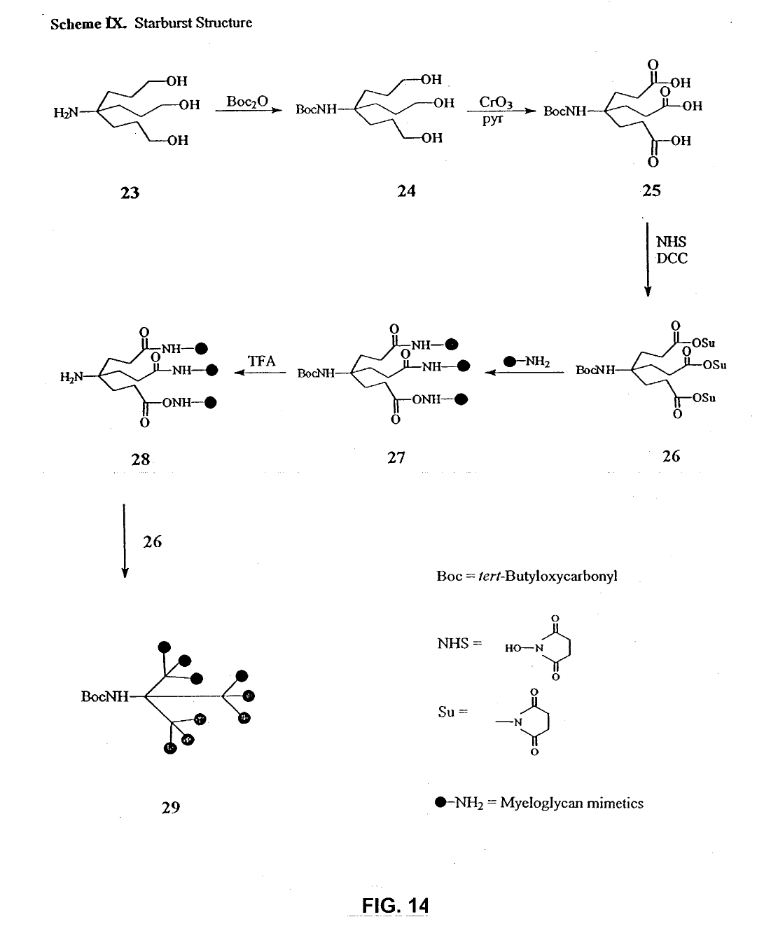

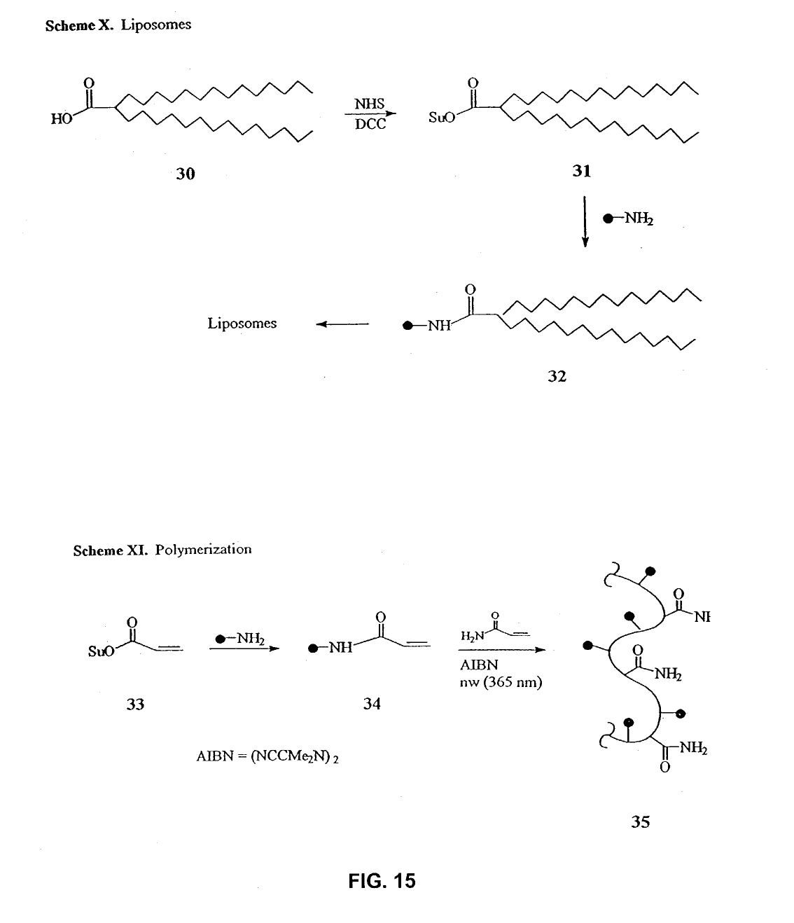

GSL's corresponding to IV³NeuAcIII³FucnLc₄Cer (SLe^{x} ceramide hexasaccharide), VI³NeuAcV³FucnLc₆Cer (SLe^{x} ceramide octasaccharide) and VI³NeuAcV³FucIII³FucnLc₆Cer (sialosyl dimeric or trimeric Le^{x} ceramide nonasaccharide), originally isolated and characterized from human tumor tissues (see Table II for structures), all are absent from the HPLC eluate of HL60 cells (FIGS. 1A and 1B). SLe^{a} also is not found in HL60 or neutrophil extracts. Instead, the entire E-selectin binding activity is associated with a series of slow-migrating components (FIG. 1B). E-selectin binding patterns of GSL's from HL60 cells and human neutrophils are identical (FIG. 2).

No binding activity was detected for ACFH-18 antigen (12) (Table III), which has 12 sugar residues, a 10-sugar backbone, five N-acetyllactosamine subunits and the VIM-2 epitope as the terminal structure.

The shortest E-selectin-binding GSL from HL60 cells was purified and characterized as having the same backbone structure as ACFH-18 antigen, but with one more internal fucosyl residue. Thus, the E-selectin-binding GSL with the shortest carbohydrate chain was eluted at a position corresponding to ceramide-tridecasaccharide (13 sugar residues).

An analogous situation was found for fraction 13-0 and fraction 14. Both contain a backbone of 12 sugars with six N-acetyllactosamine subunits. Fraction 13-0 has the VIM-2 epitope as the terminal structure and does not bind to E-selectin. Fraction 14 has the same basic structure as 13-0, but contains one or two extra internally α1→3 fucosylated residues and binds strongly to E-selectin.

The basis of structures 5-8 in Table III is as follows. (i) Each of the structures, after treatment with sialidase, does not react with anti-Le^{x} mAb SH1, but reacts strongly with anti-LacNAc mAb 1B2. Since 1B2 does not react with Le^{x}, the results indicate that each of the structures contains a sialosyl-LacNAc terminus (NeuAcα2→3Galβ1→4GlcNAcβ1→3Galβ1→R) but does not contain an SLe^{x} terminus (NeuAcα2→3Galβ1→4[Fucα1→3]GlcNAcβ1→3Galβ1→R). (ii) Each of the structures, after desialylation, reacts strongly with mAb PL82G2, which defines the structure Galβ1→4[±Fuα1→3]GlcNAcβ1→3Galβ1→4[Fucα1→3]GlcNAcβ1→3Galβ1→4[±Fucα1→3]GlcNAc→. (iii) Since the E-selectin-binding components (fractions 13-1 and 14) showed much higher levels of Fucα1→3GlcNAc residue than did the non-binding components (fractions 12 and 13-0) on ¹H-NMR (FIG. 4), but do not contain an SLe^{x} terminus, the minimal requirement for the binding structure is:

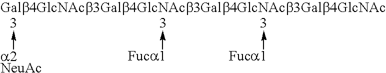

(iv) The structures cross-react strongly with anti-SLe^{x} mAb's, such as CSLEX, FH6, SNH3 and SNH4. (v) Sulfate groups were undetectable on Azure A staining on TLC.

Myeloglycans are found at the surface of neutrophils, other leukocytes and HL60 cells. Myeloglycans are found not only linked to ceramide, a sphingolipid, bound to cell membranes, but also can be linked to a carrier molecule, via, for example, a hydroxyl group. For example, the hydroxyl group may be that of serine or threonine residues of various cell membrane proteins or transmembrane proteins, such as those having a mucin-like domain, that is, having multiple repeats of a serine-rich or threonine-rich peptide. Multiple myeloglycan chains can be linked to such mucin-like core structures.

A typical example of a myeloglycan chain assembly in O-linked carbohydrate structures is:

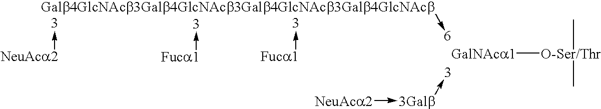

Multiple myeloglycans (MG) can be linked to a mucin-like core structure as follows:

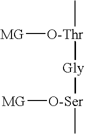

From that data derives the conclusion that the E-selectin ligand is at least a undecasaccharide bearing a terminal sialyl group and wherein at least two internal N-acetyl glucosamine (GlcNAc) residues are fucosylated. The most terminal GlcNAc residue, the penultimate GlcNAc of the backbone, is not fucosylated but as the backbone is lengthened, other internal GlcNAc residues can carry a fucosyl residue. While the size of the backbone is variable and may range to 40 residues or more, a suitable, size to the backbone is from 8 to about 22 residues, wherein the backbone comprises multiple, polymerized N-acetyllactosamine subunits.

A suitable backbone size of a myeloglycan is one containing 4 to 6 N-acetyllactosamine units and with 2 or 3 α1→3 fucosyl residues because of easier purification or synthesis, however, higher levels of binding to E-selectin may be obtained with myeloglycans with longer backbone chain lengths. The ligand of P-selectin may vary somewhat from that of the E-selectin in terms of the number of N-acetyllactosamine units and fucosyl residues.

The sialyl and fucosyl residues, and the location thereof, provide the myeloglycan with the proper charge and configuration suitable for interacting with selectin.

The instant invention contemplates a second class of myeloglycans which carry the same characteristics of the class of myeloglycan described hereinabove except that the penultimate glucosamine is fucosylated. One or more other internal residues are fucosylated as well. The conditions for the length of the backbone as for the first class of molecules applies to the second class as well. Hence, the second class of myeloglycans has a minimal structure for binding to E-selectin the following backbone:

Galβ4GlcNAc⊖3Galβ4GlcNAcβ3Galβ4GlcNAcβ3Galβ4GlcNAc

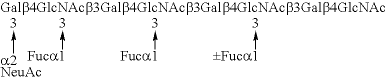

Thus, the following structures bind to E-selectin under static conditions:

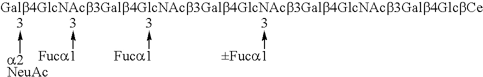

The instant myeloglycans can be synthesized also using enzymes and suitable substrates or via a series of chemical steps.

The α1→3 fucosyltransferase (FT) from HL60 cells (myeloid type FT IV) (27,28) can create an α1→3Fuc linkage at the penultimate and internal GlcNAc's of PLA to form Le^{x}, dimeric Le^{x} (Le^{x}-Le^{x}) or trimeric Le^{x} (Le^{x}-Le^{x}-Le^{x}). However, the myeloid type FT IV cannot synthesize effectively SLe^{x}, i.e. create an α1→3Fuc linkage at the penultimate GlcNAc when the terminal α2→3 sialic acid is present. Under certain conditions, the myeloid type FT IV is capable of preferentially transferring an α1→3Fuc to an internal GlcNAc (29).

The α1→3FT capable of transferring Fuc to the penultimate GlcNAc when the terminal Gal is α2→3 sialylated has been clearly distinguished from myeloid type FT IV and is identified as α1→3 FT VII (30, 31).

The FT VII of neutrophils and HL60 cells may be active enough to synthesize internal fucosylated GlcNAc residues.

Thus, a myeloglycan can be synthesized using a 2→3 sialyl transferase and fucosyltransferases and a suitable backbone structure comprising at least eight sugar residues.

There are chemical synthetic schemes which can be used to synthesize native myeloglycan. The schemes provided herein are directed to making myeloglycan derivatives. Those schemes are applicable to synthesizing wild-type myeloglycan by substituting reactants for making the naturally occurring sugar for those used to obtain a derivative.

A molecular linking group or tether can be attached to the reducing terminal of myeloglycan or derivatives thereof so that the molecules can be incorporated further to form multivalent structures, for example, by use of a starburst structure, liposomes or polymerization. A suitable tether or linking molecule is one which is bifunctional, carrying at one end a group reactive at least with GlcNAc of the myeloglycan backbone and at the opposite end of the linking molecule another generally reactive group. For example, a suitable linking molecule is a linear molecule carrying a reactive hydroxyl group at one end for reactivity with the GlcNAc residue and at the other end an amino group.

The chemical synthesis means for making myeloglycan also afford the opportunity to modify myeloglycan to obtain derivatives with desirable features, such as stability or enhanced reactivity. Derivatization of myeloglycans is constrained by the spatial relationship of the relevant substituents of the native myeloglycan, that is, a terminal sialyl residue and multiple fucosyl residues except at the penultimate GlcNAc residue.

The derivatives are designed, for example, to enforce metabolic stability of myeloglycan without affecting the ability thereof to interact with selecting. Extensive structure-function studies on sialosyl Le^{x} (SLe^{x}), which originally was thought to be a ligand for selectins, indicate that the structural elements required for SLe^{x}-selectin binding are the carboxylate group of sialic acid and the three vicinal hydroxyls of fucose (33). Therefore, the approach to construct derivatives is based, in part, on the replacement of a fucosyl residue by other functional groups, such as the more stable CF₃ analogue of a fucosyl residue (CF₃-Fuc), a 5-thio-fucosyl residue (5-S-Fuc), a 1-thio-fucosyl residue (1-S-Fuc) or a carba-fucosyl residue (C-Fuc) (FIG. 5). In addition, the sialosyl residue can be substituted for by simple anionic functional groups, including, for example, a carboxyl group, a sulfate group or a phosphate group.

Since oligolactosamine constitutes the core structure of myeloglycan, a suitable starting material is the lactosamine derivative 4, which can be prepared from a known disaccharide (34) 1 by sequential boron trifluoride etherate (BF₃-Et₂O) -induced thioglycosidation (35) (→2), deacetylation (→3) and stannylene-mediated regioselective allylation (36) (→4) (FIG. 6).

Protected Le^{x} trisaccharide derivatives can be prepared form starting material 4. The 3-OH group of lactose and N-acetyllactosamine are known to be involved in intramolecular hydrogen bonding with 5′-O (37) which results in a decreased reactivity of that OH group(38). Thus, reaction of 4 with 3 molar equiv. of benzoyl chloride (BzCl) at low temperature (−45° C.) yields the pentabenzoate 5, whereas the conventional benzoylation affords the hexabenzoate 6 (FIG. 7).

The requisite glycosyl donor 7 for derivative preparation is obtained from CF₃-Fuc (39) according to the procedure employed for fucose (40) which involves 1) formation of methyl α-glycoside, 2) benzylation, 3) acid hydrolysis and 4) trichloroimidation. The synthesis of other. glycosyl donor 8 has been reported. (41)

Stereoselective α-glycosylation of 5 with 7-and 8 proceeds effectively in the presence of BF₃-Et₂O to produce 9 and 10, respectively. In the case of 5-thioglycosylation, it is reported that the 1,2-cis glycoside predominates even in the presence of the 2-O-acetyl group.(41)

1-S-Fuc and C-Fuc are introduced by substitution reactions of triflate using 14 (42) and 15 (43) as nucleophiles, respectively. FIG. 8 summarizes the preparation of the triflate 13, which involves the epimerization of the 3-OH group in 5 by an oxidation (→11) and reduction (→12) sequence.

After generation of a thiolate (from 14) and an alcoholate (from 15) by treatment with sodium hydride, substitution reaction of 13 leads to the formation of 16 and 17, respectively (FIG. 9).

The aminohexyl linking molecule or tether is introduced to a reducing terminal of each Le^{x} trisaccharide derivative 9, 10, 16 or 17 by glycosylation of 18 (44) using methyl trifluoromethanesulfonate (MeOTf) (45) as a promoter (FIG. 10).

With the monomeric Le^{x} derivatives (9, 10, 16 and 17) and those with a linking molecule or tether (19-22) readily available, the dimeric framework is assembled as shown in FIG. 11. Selective removal of the allyl protecting group 19-22 through isomerization with (Ph₃P)₃RhCl leads to the 3′-OH disaccharides which react with glycosyl donor 9/10/16/17 in the presence of MeOTf affording the corresponding dimeric Le^{x} derivatives.

Reiteration of the deallylation and coupling procedures leads to the corresponding trimeric derivatives (FIG. 11). Further deallylation of the trimers provides the proper acceptors for the next glycosylation.

FIG. 12 provides the continuation of the buildup toward tetralactosamine core B. Thus, glycosylation of trimeric Le^{x} derivative A of FIG. 11 with 6 is followed by dephthaloylation using hydrazine hydrate, which concomitantly removes acyl protecting groups, and subsequent N, O-acetylation affords B. Selective removal of the allyl protecting group from B furnishes monohydroxyglycoside C.

The allyl functionality in B is transformed to a carboxyl group either by ozonolysis or by a hydroboration and oxidation sequence (46) (FIG. 13). On the other hand, sulfated and phosphorylated analogues can be prepared from C. Thus, exposure of C to the SO₃.NMe₃ complex in anhydrous pyridine (47) provides the sulfated derivatives, and phosphorylation of C by phosphitylation with dibenzyl N,N-diisopropylphosphoramidite and 1H-tetrazole, followed by oxidation with 3-chloroperoxybenzoic acid (m-CPBA) (48), affords the phospholylated derivatives.

Finally, deacetylation of the protected derivatives followed by hydrogenolysis yields the target myeloglycan derivatives.

The myeloglycan derivatives can be manipulated further through an amino functionality of the linking groups or tethers.

For example, oxidation of the trifunctional molecule 24 obtained from commercially available tris(3-hydroxypropyl)aminomethane (23) to the tris(carboxylic acid), followed by esterification with N-hydroxysuccinimide, yields the tris(active ester) 26 (FIG. 14). A typical coupling reaction between 26 and myeloglycan derivatives provides the trivalent derivative 27. The Boc group can be cleaved by acidolysis for further derivation to starburst structures.

To obtain liposomes, commercially available 2-tetradecylhexadecanoic acid (30) is converted into the active ester 31 and then coupled to myeloglycan derivatives (FIG. 15, top). The resulting neoglycolipid 32 is used to prepare a liposome using known techniques. (55)

Free-radical polymerization of the acrylamide derivative 34, prepared from 33 and myeloglycan derivatives, with acrylamide results in formation of the copolymer 35 in which composition and structure can be varied readily (FIG. 15, bottom).

Various modifications also can be made to the myeloglycan backbone. Moreover, changes to the backbone, as will be described hereinbelow, and the changes to the relevant substituents described hereinabove, can be combined in a single derivative molecule.

A pharmacophore search can be used to find alternative backbone structures, which may or may not comprise saccharide, which can be used to configure or identify a molecule which binds selectin can be obtained. First the myeloglycan pharmacophore is identified by structure-function studies, as described, for example, in the studies directed to SLe^{x}. (56) Distance parameters of the resulting functional groups are defined by use of NMR data, such as Nuclear Overhauser Effect (NOE), spectroscopy, methylation analysis and the like, coupled with conformational energy computations.

Based on the results of such physical studies, a minimum energy conformation model of myeloglycan can be obtained by computer assisted modeling, a number of software programs are known in the art. For example, a myeloglycan model was constructed based on HSEA (Ha_d Sphere Exo-Anomeric) calculations with the GESA (Geometry of Saccharides) program (Dr. Bernd Meyer, Department of Biochemistry, University of Georgia, Athens, Ga.) and visualized using the SYBYL molecular graphics program (Tripos Associates, St. Louis, Mo.) with computations performed on a Silicon Graphics IIRIS 4D/85 system (57).

The modeling demonstrated that the repeating N-acetyllactosamine core forms a helical structure with the carboxylate of sialic acid and the three vicinal hydroxyls of the internal fucose residues presented on that longitudinal structure in a specific spatial relationship. The conformation comprises the following glycosidic torsion angles (Φ/Ψ): NeuAcα2→3Gal (−170°/−7°), Galβ1→4GlcNAc (54°/9°), Fucα1→3GlcNAc (49°/24°), GlcNAcβ1→3Gal (57°/−10°) and Galβ1→4Glc (55°/2°).

The spatial dispositions of those functional groups are used to construct a model and a pharmacophore. Against that template, a synthetic molecule comprising a polymer or a single monomer can be substituted for the polylactosamine backbone or molecule per se carrying the appropriate charges, hydrophobicity and the like of the relevant backbone elements and substituents in the same spatial organization as found in the native molecule to enable interaction of the substitute with selectin.

Alternatively, a search of available molecules approaching or having the necessary physical characteristics may reveal one, which although chemically unrelated, nevertheless may function as a substitute for the myeloglycan backbone or myeloglycan per se.

For example, the Fine Chemicals Directory data base (FCD 91.1) can be searched using the MACCS-3D software (Molecular Designs, Ltd., San Leandro, Calif.). Compounds are screened initially in the 2-D mode and matched compounds then are evaluated in the 3-D mode. Lead compounds then are subjected to biologic evaluation to select those with greatest impact on selectin binding. The lead compounds are modified, as described hereinabove, for example, to maximize selectin inhibition. That very approach was applied to SLe^{x} and various non-carbohydrate inhibitors, such as a terpenoid compound, were obtained which successfully substitute for SLe^{x} in biologic and functional assays. (56)

Because the isolated myeloglycans are novel structures, the molecules can be used to generate antibodies thereto, which may be employed within the context of the instant invention to block the selectin-ligand binding reaction or for use as reagents for detecting myeloglycans. As to the various possible uses of myeloglycans, either a native myeloglycan or a derivative thereof may be used. As used herein, such antibodies include both monoclonal and polyclonal antibodies and may be intact molecules, a fragment of such a molecule or a functional equivalent thereof retaining binding specificity. The antibody may be engineered genetically. Examples of antibody fragments include F(ab′)₂, Fab′, Fab and Fv fragments.

Briefly, polyclonal antibodies are produced by immunizing an animal and subsequent collection of serum therefrom. Immunization is accomplished, for example, by a systemic administration, such as by subcutaneous, intrasplenic or intramuscular injection, into a rabbit, rat or mouse. It is preferred generally to follow the initial immunization with one or more booster immunizations prior to serum collection. Such methodology is well known and described in a number of references.

While polyclonal antibodies may be employed in the instant invention, monoclonal antibodies also are suitable. Monoclonal antibodies suitable for use within the instant invention include those of murine or human origin, or chimeric antibodies such as those which combine portions of both human and murine antibodies (i.e., antigen binding region of murine antibody plus constant regions of human antibody). Human and chimeric antibodies are produced using methods known by those skilled in the art. Human antibodies and chimeric human-mouse antibodies are advantageous because of a theoretic reduced risk of generating xenogeneic antibodies thereto when administered clinically.

Monoclonal antibodies may be produced generally by the method of Köhler and Milstein (49 and 50), as well as by various techniques which modify the Köhler and Milstein method, see (51). Briefly, the lymph nodes and/or spleen of an animal immunized with one of the myeloglycans reactive with selectin are fused with myeloma cells to form hybrid cell lines (“hybridomas” or “clones”). Each hybridoma secretes a single type of immunoglobulin and, like the myeloma cells, has the potential for indefinite cell division. For immunization, it may be desirable to couple such myeloglycans to a carrier to increase immunogenicity. Suitable carriers include keyhole limpet hemocyanin, thyroglobulin, bovine serum albumin and derivatives thereof.

An alternative to the production of monoclonal antibodies via hydridomas is the creation of monoclonal antibodies expression libraries using bacteriophage and bacteria, see, for example, (52) and (53), or by in vitro immunization. Selection of antibodies exhibiting appropriate specificity may be performed in a variety of ways which will be evident to those skilled in the art.

A suitable antibody with specificity for a myeloglycan which binds selectin can be used as a reagent for detecting same in any of a variety of art-recognized assay formats, such as RIA, ELISA and an assay monitored in a flow cytometer. Essentially a sample is exposed to the myeloglycan antibody. The myeloglycan antibody can be labelled. If labelled, following wash, presence of bound antibody is ascertained using an appropriate detector, such as scintillation counter or X-ray film for a radio-labelled antibody or a spectrophotometer for an enzyme-labelled antibody following exposure to a suitable substrate. If not labelled, a suitable second antibody is used, which second antibody may be labelled.

Obtention of purified sources of myeloglycans provides a method for inhibiting cell aggregation, immune cell aggregation, platelet aggregation and the like within a biologic preparation wherein aggregation is reliant on interaction of myeloglycan and selectin. The method comprises incubating a biologic preparation with at least one myeloglycan.

Purified or synthesized myeloglycan is precipitated, dialyzed to remove unwanted reagents and suspended in a physiologic buffer prior to use. The myeloglycan solution can be treated to provide a dry preparation, such as a powder, by lyophilization, for example.

Suitable biologic preparations include cell cultures and cell suspensions in biological fluids, such as blood, urine, lymph, synovial and cerebrospinal fluid. Myeloglycans generally will be incubated at a final concentration of about 0.1 to 1 M, and typically at about 0.2 to 0.5 M. Incubation is performed typically for 5 to 15 minutes at 37° C.

The instant invention also provides a method for inhibiting unwanted cell aggregation in a warm-blooded animal, such as a human. The method comprises administering to a warm-blooded animal an effective amount of at least one myeloglycan, the myeloglycan inhibiting the binding of cells to sites expressing selectin. The instant myeloglycans can function as an anti-inflammatory agent.

The myeloglycans generally will be administered at a concentration of about 0.1 to 1 M and typically at about 0.2 to 0.5 M. It will be evident to those skilled in the art how to determine the optimal effective dose for a particular substance, e.g., based on in vitro and in vivo studies in non-human animals. A variety of routes of administration may be used. Typically, administration will be intravenous, intramuscular or intracavitary, e.g., in the pleural or peritoneal cavities, in the bed of a site of inflammation.

A myeloglycan can be combined with any of a variety of known excipients, fillers and the like known in the pharmaceutic arts as non-critical ingredients of a drug formulation aimed at enhancing properties of the final product. Any of a variety of standard pharmaceutic texts can be consulted, such as Remington's.

The myeloglycans also can be delivered by alterative means, such as by infusion pump, implant, patch, topically, by depot and the like. The myeloglycans can be contained within microspheres, such as microcapsules and liposomes. Standard methods for preparing same are known in the art (55).

Moreover, myeloglycan may be administered in combination with an immunotherapeutic or chemotherapeutic substance or in combination with an anti-inflammatory substance. When a combination of a myeloglycan and a substance is desired, each compound may be administered sequentially, simultaneously or combined and administered as a single composition. Dosages of each active ingredient are adjusted according to data obtained in vitro, animal studies or empirical clinical studies, as is known in the art.

Diagnostic techniques, such as CAT scans, may be performed prior to and subsequent to administration to confirm the effectiveness of the inhibition of metastatic potential or inflammatory potential.

The instant invention now will be exemplified in the following non-limiting examples.

### EXAMPLES

### Example 1

HL60 cells were obtained originally from the American Type Culture Collection (ATCC) and grown in RPMI supplemented with 15% FCS. Cells were cultured continuously in roller bottles and harvested every four days. Altogether, 1100 mL of packed HL60 cells were divided into ≈300 mL packed aliquots. Normal (non-leukemic) human leukocytes (mostly neutrophils) were obtained from Japan Immunoresearch Laboratories, Takasaki City, Japan, wherein the cells were collected using an ex vivo circulatory system with a specific adhesion column. Frozen neutrophils were subjected directly to extraction of polar GSL's.

CHO cell tranfectants with E-selectin and P-selectin cDNA were established as follows. E-selectin cDNA in pCDM-8 was obtained from R&D Systems, Minneapolis Minn. P-selectin cDNA was cloned from HEL cells (ATCC) based on the published sequence (2) and ligated in pRC/CMV (InVitrogen, San Diego Calif.). Chinese hamster ovary (CHO) DG44 cells (Dr. L. A. Chasin, Columbia University, NY) were cotransfected with E-selectin/pCDM-8 or P-selectin/pRC/CMV with pSV2/dhfr (ATCC) as described previously (10). The transfected genes were amplified by stepwise selection for resistance to increasing concentrations of methotrexate (up to 3 μM and 5 μM for P-selectin and E-selectin expressors, respectively). P-selectin and E-selectin-expressing clones were isolated by cytofluorometry using anti-P-selectin mAb, such as, P11A, and anti-E-selectin mAb, such as, E12. The mAb's were established through immunization of BALB/c mice with NS-1 cells expressing P-selectin or E-selectin by standard procedures.

### Example 2

Frozen cell pellets were extracted in five volumes of IHW (55:50:25 v/v/v) in a Waring blender for 5 min and suction filtered through Celite (Fisher Chemical Co.). The extraction was repeated three times.

Extracts were combined and evaporated to dryness under reduced pressure, the residue was dissolved in one volume water and Folch partitioned with six volumes of CM, 2:1. The lower phase was repartitioned three times with theoretical upper phase. Upper phases were combined, evaporated to a small volume (≈10 mL), dialyzed in distilled water through a Spectropore 5000 dialysis tubing and lyophilized.

The residue was dissolved in CMW 1:10:10 and applied to diethylaminoethyl Sephadex, as described previously (11). The neutral GSL fraction present in pass-through, monosialoganglioside fraction eluted with the same solvent containing 0.03 M ammonium acetate and disialoganglioside fraction eluted with the same solvent containing 0.13 M. ammonium acetate were separated. Each fraction was concentrated, dialyzed and lyophilized.

The monosialoganglioside fraction was dissolved in IHW (55:40:5), introduced into an Iatrobead™ column and subjected to gradient elution with IHW, as described in the legend of FIG. 1. A similar elution program was used previously for separation of monosialogangliosides (12, 13). IV³NeuAcnLc₄Cer, VI³NeuAcnLc₆Cer, IV⁶NeuAcnLc₄Cer, VI⁶NeuAcnLc₆Cer, IV³NeuAcIII³FucnLc₄Cer (SLe^{x} ceramide hexasaccharide), VI³NeuAcVFucnLc₆Cer (SLe^{x} ceramide octasaccharide) and VI³NeuAcV³FucIII³FucnLc₆Cer (sialosyl dimeric Le^{x} ceramide nonasaccharide) were eluted at defined positions as shown by the arrows in FIG. 1. Further purification of the E-selectin-binding GSL fraction was performed by acetylation and separation on preparative HPTLC as described previously (12, 13). Separated fractions were deacetylated in CM-1% sodium methoxide in methanol, 2:1:0.1, for 10 min and desalted using known techniques.

### Example 3

GSL fractions separated by HPLC as described herein were analyzed by HPTLC developed in various polar solvents (see legend of FIGS. 1 and 2). The TLC plate was blotted with metabolically ³²P-labeled CHO cells expressing E-selectin or P-selectin as described previously (14) (see FIG. 1 legend).

### Example 4

To determine whether the GSL in question has the SLe^{x} structure or NeuAcα2→3Galβ1→4GlcNAcβ1→3Galβ1→ structure, GSL's were desialylated by sialidase followed by TLC and then immunostaining with anti-Le^{x} mAb's (e.g., SH1, FH2, anti-SSEA-1) or by immunoblotting with mAb 1B2 (which does not react with Le^{x} but does react with the LacNAc terminus Galβ1→4GlcNAcβ1→3Galβ1→R). The procedure is described in the FIG. 3 legend.

### Example 5

The reactivity of each fraction was tested before and after sialidase treatment with mAb PL82G2 which binds to internally located Fucα1→3GlcNAc and various antibodies directed to SLe^{x} such as FH6 (15), CSLEX (16), SNH3 and SNH4.

### Example 6

Sulfate group was detected on TLC with the cationic dye, Azure A, as described previously (17, 18). Sodium chlorate, which blocks biosynthesis of sulfate from PAPS was used to detect HL60 cell adhesion to E-selectin.

### Example 7

¹H-NMR spectra were recorded with a Bruker AM-500 spectrometer equipped with an Aspect 3000 computer and pulse programmer, operating in the Fourier transform mode with quadrature detection. Spectra were recorded at 328° K (for ACFH-18 antigen) or 325° K (for myeloglycan GSL fractions 13 and 14) (19) on deuterium-exchanged samples dissolved in 0.4 mL of dimethyl-sulfoxide-d₆ containing 2% D₂O (20) and 1% tetramethylsilane as a chemical shift reference. Other parameters and data treatment were as described previously (19).

### Example 8

As disclosed in (58)-(60), SLe^{x} can affect cell aggregation in various animal models. In similar fashion, myeloglycan can be shown to intervene in cell aggregation.

The highly metastatic BL6 clone of the B16 melanoma cell line (Dr. Jean Starkey, Montana State Univ., Bozeman, Mont.) was selected in syngeneic C57BL mice for high metastatic potential. C57BL mice were maintained in plastic cages under filtered air atmosphere and provided with water and food pellets. Cells were cultured in RPMI 1640 supplemented with 2 mM glutamine and 10% fetal calf serum (FCS) and detached with phosphate buffered saline (PBS) containing 2 mM EDTA. Viability was tested by trypan blue exclusion test.

A suspension of BL6 cells (1-3×10⁶ cells/ml RPMI 1640 medium) was prepared and aliquots are incubated in the presence or absence of myeloglycans at various concentrations, at 37° C. for 5-10 minutes. Following incubation, typically, 3×10⁴ or 2×10⁴ cells (with or without myeloglycan pretreatment) per 200 μl are injected via a tail vein into 8-week-old female mice. After 18-21 days, the mice are killed, the lungs are fixed in 10% formaldehyde in PBS (pH 7.4) and tumor cell colonies are counted under a dissecting microscope. Data on the number and the size of colonies are treated statistically by the analysis of variance (ANOVA) procedure. Colonies with a diameter of 1 mm or greater are considered large-size and those with a diameter less than 1 mm are considered small-size.

Colony number is reduced in animals receiving cells exposed to myeloglycan.

### Example 9

Mice are exposed to radiolabelled myeloglycan by intravenous injection. Myeloglycan is radiolabelled using known synthesis methods such as using a radiolabelled starting material as disclosed in the synthetic schemes described herein. For example, tritiated or ¹⁴C-labelled fucose or a fucose analog carrying ³⁵S can be used to label a myeloglycan. Varying amounts of labelled myeloglycan are administered to a host animal. Then any of a variety of known models of leukocyte adherence to endothelium can be used to provide a site for selectin expression, see Table 6.2 and references cited therein for a list of experimental models of vascular and tissue injury in (54).

Localization of labelled myeloglycan at the injury site can be assessed using known methods. Assessments can be taken at varying time points. Also, serum levels of myeloglycan can be ascertained. Such data will yield a suitable dose regimen to assure localization of adequate myeloglycan at the injury site.

Unlabelled myeloglycan at the thus empirically determined dose is administered to experimental hosts. The injury to obtain selectin expression is induced and then metabolically labelled leukocytes or tumor cells are administered to the treated host. The cells are labelled, for example, by culture in the presence of a radiolabelled nutrient, such as ³⁵S methionine. The degree of labelled cell binding to the injury site is assessed using known techniques.

Binding of leukocytes and transformed cells to the injury site is reduced in animals pre-treated with myeloglycan.

### REFERENCES

1. Bevilacqua, M. P., et al. (1989) Science 243, 1160-1165.

2. Johnston, G. I. et al. (1989) Cell 56, 1033-1044.

3. Lowe, J. B. et al. (1990) Cell 63, 475-484.

4. Phillips, M. L. et al. (1990) Science 250, 1130-1132.

5. Berg., E. L. et al. (1991) J. Biol. Chem. 266, 14869-14872.

6. Takada, A., et al. (1991) Biochem. Biophys. Res. Commun. 179, 713-719.

7. Handa, K. et al. (1991) Biochem. Biophys. Res. Commun. 181, 1223-1230.

8. Tiemeyer, M. et al. (1991) Proc. Natl. Acad. Sci. USA 88, 1138-1142.

9. Lowe, J. B. et al. (1991) J. Biol Chem. 266, 17467-17477.

10. Ito, K. et al. (1994) Glycoconj. J. 11, 232-237.

11. Yu, R. K. & Ledeen, R. W. (1972) J. Lipid Res. 13, 680-686.

12. Nudelman, E. D. et al. (1988) J. Biol. Chem. 263, 13942-13951.

13. Nudelman, E. D. et al. (1989) J. Biol. Chem. 264, 18719-18725.

14. Swank-Hill et al. (1987) Anal. Biochem. 163, 27-35.

15. Fukushi, Y. et al. (1984) J. Biol. Chem. 259, 10511-10517.

16. Fukushima, K. et al. (1984) Cancer Res. 44, 5279-5285.

17. Iida, N. et al. (1989) J. Biol. Chem. 264, 5974-5980.

18. Schnaar, R. L. & Needham, L. K. (1994) Meth. Enzymol. 230, 371-389.

19. Levery, S. B. et al. (1986) Carbohydr. Res. 151, 311-328.

20. Dabrowski, J. et al. (1980) Biochemistry 19, 5652-5658.

21. Fukushi, Y. et al. (1984) J. Exp. Med. 159, 506-520.

22. Symington, F. W. et al. (1985) J. Immunol. 134, 2498-2506.

23. Stroud, M. R. et al. (1994) Biochemistry 33, 10672-10680.

24. Fukuda, M. et al. (1984) J. Biol. Chem. 259, 10925-10935.

25. Fukuda, M. et al. (1985) J. Biol. Chem. 260, 12957-12967.

26. Asada, M. et al. (1991) Biochemistry 30, 1561-1571.

27. Potvin, B. et al. (1990) J. Biol. Chem. 265, 1615-1622.

28. Goelz, S. E. et al. (1990) Cell 63, 1349-1356.

29. Holmes, E. H. & Macher, B. A. (1993) Arch. Biochem. Biophys. 301, 190-199.

30. Sasaki, K. et al. (1994) J. Biol. Chem. 269, 14730-14737.

31. Natsuka, S. et al. (1994) J. Biol. Chem. 269, 16789-16794.

32. Levery, S. B. et al. (1988) Carbohydr. Res. 178, 121-144.

33. (a) Tyrrell, D. et al. (1991) Proc. Natl. Acad. Sci. U.S.A. 88, 10372-10376. (b) Graves, B. J. et al. (1994) Nature 367, 532-538. (c) Ramphal, J. Y. et al. (1994) J. Med. Chem. 37, 3459-3463.

34. Arnarp, J. & Lönnqren, J. (1981) J. Chem. Soc.

Perkin I, 2070-2074.

35. Dahmén, J. et al. (1983) Carbohydr. Res. 114, 328-330.

36. Alais, J. et al. (1983) Tetrahedron Lett., 24, 2383-2386.

37. Hirotsu, K. & Shimada, A. (1974) Bull. Chem. Soc. Jpn. 47, 1872-1879.

38. (a) Bhatt, R. S. et al. (1977) J. Chem. Soc. Perkin I, 2001-2005. (b) Nashed, M. A. & Musser, J. H. (1993) Carbohydr. Res., 250, C1-C4.

39. Bansal, R. C. et al. (1991) J. Chem. Soc., Chem. Commun., 796-798.

40. Wegmann, B. & Schmidt, R. R. (1988) Carbohydr. Res. 184, 254-261.

41. Hashimoto, H. & Izumi, M. (1993) Tetrahedron Lett. 34, 4949-4952.

42. Hashimoto, H. et al. (1993) Tetrahedron Lett. 34, 4953-4956.

43. Cai, S. et al. (1992) J. Org. Chem. 57, 6693-6696.

44. Ammann, H. & Dupuis, G. (1988) Can. J. Chem. 66, 1651-1655.

45. Lönn, H. (1985) Carbohydr. Res. 139, 105-113.

46. Brown, H. C. et al. (1992) J. Org. Chem. 57, 6173-6177.

47. Nicolaou, K. C. et al. (1993) J. Am. Chem. Soc. 115, 8843-8844.

48. Yum, K. -L. & Fraser-Reid, B. (1988) Tetrahedron Lett. 29, 979-982.

49. Kohler & Milstein (1975) Nature 256, 495-497.

50. Kohler & Milstein (1976) Eur. J. Immunol. 6, 511-519.

51. Harlow & Lane, eds. (1988) “Antibodies: A Laboratory Manual” Cold Spring Harbor, N.Y.

52. Sastry et al. (1989) Proc. Natl. Acad. Sci. 86, 5728.

53. Huse et al. (1989) Science 246, 1275.

54. Harlan et al. (1992) in “Adhesion” Harlan & Liu, eds., Chap. 6, Freeman & Co., NY.

55. Park & Huang (1993) Biochim. Biophys. Acta 1166, 105-114.

56. Rao, B. et al. (1994) J. Biol. Chem. 269, 19663-19666.

57. Kojima, N. et al. (1994) Glycoconjugate J. 11, 238-248.

58. Winn et al. (1993) J. Clin. Invest. 92, 2042-2047.

59. Mulligan et al. (1992) J. Clin. Invest. 90, 1600-1607.

60. Mulligan et al. (1993) J. Exp. Med. 178, 623-631.

All references cited herein are incorporated by reference in entirety.

An artisan will well recognize that various changes and modifications can be made to the teachings of the instant specification without departing from the spirit and scope of the instant invention.

TABLE 1
Functional group analysis of GSL fractions by mAbs and ¹H-NMR.

| | mAb | | | | αFuc1 → 3- | Fuc-5 quartet | | | GlcNAc-1 doublet | | |
|---|---|---|---|---|---|---|---|---|---|---|---|
| | 1B2 | SH1 | | FH6 | GlccNAc | 4.590 | 4.594 | 4.603 | 4.748 | 4.741 | 4.736 |
| Fraction | (LacNAc) | (Le^{x}) | PL82G2 | (SLe^{x}) | 4.875 ppm | ppm | ppm | ppm | ppm | ppm | ppm |
| 12-1 | − | − | − | + | | | | | | | |
| desialylated | ++ | − | + | − | | | | | | | |
| 13-1* | − | − | − | + | | | | | | | |
| desialylated | ++ | − | + | − | | | | | | | |
| 13-0 | − | − | − | + | + | + | | | | + | |
| desialylated | ++ | − | ++ | − | | | | | | | |
| 14* | − | − | − | ++ | +++ | | + | + | + | | + |
| desialylated | ++ | − | ++ | − | | | | | | | |

*Shows strong binding to E-selectin.

TABLE II
Glycoconjugates isolated and characterized from human tumors and containing SLe^{X} epitope.

| | | Presence in neutrophils |
|---|---|---|
| No. | Structure | and HL60 cells |
| 1 | 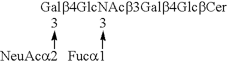 | — |
| 2 | 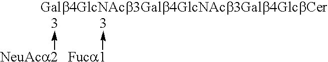 | — |
| 3 | 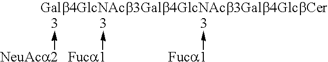 | — |
| 4 | 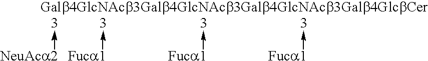 | — |

TABLE III
Major glycoconjugates present (on GSL's) in HL60 cells and human neutrophils.

| No. | Structure | E-selectin binding |
|---|---|---|
| 5 | 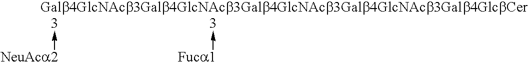 | − |
| 6 | 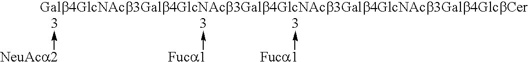 | ++ |
| 7 | 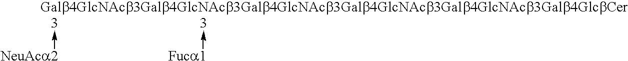 | − |
| 8 | 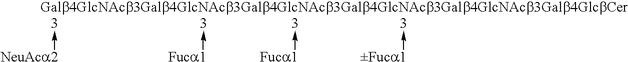 | ++ |

What is claimed is:

1. An isolated oligosaccharide of the formula: 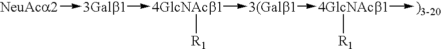
wherein R₁ is H or α1→3Fuc, provided that at least two R₁ groups are α1→3Fuc, and NeuAc is sialic acid, Gal is galactose, GlcNAc is N-acetyl glucosamine and Fuc is fucose.

2. The oligosaccharide of claim 1, wherein NeuAc is replaced with an anionic group.

3. The oligosaccharide of claim 2, wherein the anionic group is a carboxyl group, a sulfate group or a phosphate group.

4. The oligosaccharide of claim 1, wherein fucose is replaced with 5-thio fucose, 1-thio fucose, carbafucose or 6-trifluorofucose.

5. The oligosaccharide of claim 1, wherein the number of repeating N-acetyllactosamine subunits containing R₁ is 3 to 5.

6. A composition comprising an isolated oligosaccharide of the formula: 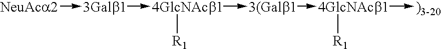
wherein R₁ is H or α1→3Fuc, provided that at least two R₁ groups are α1→3Fuc, and NeuAc is sialic acid, Gal is galactose, GlcNAc is N-acetyl glucosamine and Fuc is fucose, and an excipient or diluent.

7. The composition of claim 6, wherein the terminal GlcNAc residue of said oligosaccharide is attached to a bifunctional linking molecule.

8. The composition of claim 6, wherein said oligonucleotide is attached via the terminal GlcNAc residue and a hydroxyl group to a carrier molecule.

9. The composition of claim 8, wherein said oligonucleotide is attached to serine or threonine of said carrier.

10. The composition of claim 6, comprising a plurality of isolated oligosaccharides.

11. The composition of claim 6 which is contained in a microsphere.

12. The composition of claim 11, wherein said microsphere is a liposome.

13. The composition of claim 6, wherein said oligosaccharide comprises a liposome membrane.

14. The composition of claim 6, wherein the number of repeating N-acetyllactosamine subunits containing R₁ is 3 to 5.

15. A method for synthesizing an oligosaccharide which binds selectin comprising an unbranched backbone with a terminal galactose and a terminal N-acetylglucosamine comprising:
(a) attaching N-acetylglucosamine and galactose of N-acetyllactosamine subunits to provide said unbranched backbone comprising 4-21 N-acetyllactosamine subunits;
(b) attaching a sialic acid to said terminal galactose; and
(c) attaching a fucose to N-acetylglucosamine of N-acetyllactosamine subunits;
provided that said oligosaccharide contains at least two fucoses.

16. The method of claim 15, wherein the order of steps is (c), (a) and (b).

17. The method of claim 15, further comprising the step of attaching said oligosaccharide to a carrier at said terminal N-acetylglucosamine.

18. The method of claim 17, wherein said carrier is lipid.

19. The method of claim 17, wherein said carrier is protein.

20. The method of claim 17, wherein said carrier is a bifunctional linking molecule.
